(19) United States
(12) Patent Application Publication
(10) Pub. No.: US 2023/0240510 A1
(43) Pub. Date: Aug. 3, 2023

(54) EXAMINATION SUPPORT DEVICE, EXAMINATION SUPPORT METHOD, AND STORAGE MEDIUM STORING EXAMINATION SUPPORT PROGRAM

(71) Applicant: AI Medical Service Inc., Tokyo (JP)

(72) Inventor: Kenichirou SUZUKI, Tokyo (JP)

(21) Appl. No.: 18/131,706

(22) Filed: Apr. 6, 2023

Related U.S. Application Data
(63) Continuation of application No. PCT/JP2021/037449, filed on Oct. 8, 2021.

(30) Foreign Application Priority Data
Oct. 22, 2020 (JP) 2020-177108

(51) Int. Cl. A61B 1/00; A61B 1/045

(52) U.S. Cl. CPC A61B 1/00004; A61B 1/00055; A61B 1/045

(57) ABSTRACT

There is provided an examination support device with which, in a screening examination that uses an endoscope, an image that should be acquired can be reliably acquired and discomfort of a subject can be reduced. The examination support device includes an acquisition unit that sequentially acquires a captured image captured by a camera unit inserted in a body of a subject, a determination unit that determines whether the captured image acquired by the acquisition unit matches any of scheduled images that should be acquired, a detection unit that detects a timing of removal of the camera unit from an examination part based on the captured image acquired by the acquisition unit, and a warning unit that issues a warning in a case where there remains, at the timing detected by the detection unit, the scheduled images for which match is not determined by the determination unit.

### CROSS REFERENCE TO RELATED APPLICATION

The present application is based upon and claims the benefit of priority from Japanese Patent Application No. 2020-177108, filed on Oct. 22, 2020, and International application No. PCT/JP2021/037449 filed on Oct. 10, 2021, the entire contents of which are hereby incorporated by reference.

### BACKGROUND

### Technical Field

The present invention relates to an examination support device, an examination support method, and a storage medium storing an examination support program.

### Background Art

There is known an endoscope system that captures, for example, the inside of a stomach of a subject by an endoscope, and that displays an image of the inside of the stomach on a monitor. These days, examination support devices that analyze an image captured by an endoscope system and that notify a doctor of the result are becoming widespread (for example, see Patent Literature 1).

### CITATION LIST

### Patent Literature

[Patent Literature 1] Japanese Patent Laid-Open No. 2019-42156

### SUMMARY

### Technical Problem

Criteria are often set for screening examinations that use an endoscope such that various sections of an examination part (such as a stomach) are captured from various angles and at various angles of view to be recorded as still images. However, due to lack of experience or an error of a doctor manipulating the endoscope, a still image that should be recorded may possibly be forgotten to be taken or may be unclear. Since insertion/removal of an endoscope to/from inside a body can be somewhat uncomfortable to a subject, there is an issue that redoing of examination should be avoided as much as possible.

The present invention has been made to solve problems as described above, and provides an examination support device and the like with which, in a screening examination that uses an endoscope, an image that should be acquired can be reliably acquired and discomfort of a subject can be reduced.

### Solution to Problem

An examination support device according to a first mode of the present invention includes an acquisition unit that sequentially acquires a captured image captured by a camera unit inserted in a body of a subject; a determination unit that determines whether the captured image acquired by the acquisition unit matches any of scheduled images that should be acquired; a detection unit that detects a timing of removal of the camera unit from an examination part based on the captured image acquired by the acquisition unit; and a warning unit that issues a warning in a case where there remains, at the timing detected by the detection unit, the scheduled images for which match is not determined by the determination unit.

Furthermore, an examination support method according to a second mode of the present invention includes an acquisition step of sequentially acquiring a captured image captured by a camera unit inserted in a body of a subject; a determination step of determining whether the captured image acquired in the acquisition step matches any of scheduled images that should be acquired; a detection step of detecting a timing of removal of the camera unit from an examination part based on the captured image acquired in the acquisition step; and a warning step of issuing a warning in a case where there remains, at the timing detected in the detection step, the scheduled images for which match is not determined in the determination step.

An examination support program according to a third mode of the present invention causes a computer to perform: an acquisition step of sequentially acquiring a captured image captured by a camera unit inserted in a body of a subject; a determination step of determining whether the captured image acquired in the acquisition step matches any of scheduled images that should be acquired; a detection step of detecting a timing of removal of the camera unit from an examination part based on the captured image acquired in the acquisition step; and a warning step of issuing a warning in a case where there remains, at the timing detected in the detection step, the scheduled images for which match is not determined in the determination step.

### Advantageous Effect of Invention

According to the present invention, there can be provided an examination support device and the like with which, in a screening examination that uses an endoscope, an image that should be acquired can be reliably acquired and discomfort of a subject can be reduced.

### BRIEF DESCRIPTION OF DRAWINGS

FIG. 1 is a diagram showing a manner of an endoscopic examination performed using an endoscope system and a stomach examination support device according to a present embodiment.

FIG. 2 is a hardware configuration diagram of the stomach examination support device.

FIG. 3 is a diagram describing a process up to generation of a display screen from a display signal that is received.

FIG. 4 is a diagram describing a change in a captured image that is reproduced.

FIG. 5 is a diagram describing a process of match determination.

FIG. 6 is a diagram describing a process of match determination.

FIG. 7 is a diagram describing a process of removal detection.

FIG. 8 is a diagram describing another process of removal detection.

FIG. 9 is a diagram describing further another process of removal detection.

FIG. 10 is an example of a warning screen that is displayed in a case where there remains a scheduled image.

FIG. 11 is a flowchart describing a procedure of an arithmetic processing unit.

### DESCRIPTION OF EMBODIMENTS

Hereinafter, the present invention will be described based on an embodiment of the invention, but the invention in the scope of the claims is not limited to the embodiment described below. Moreover, not all the configurations described in the embodiment are essential as means for solving the problems.

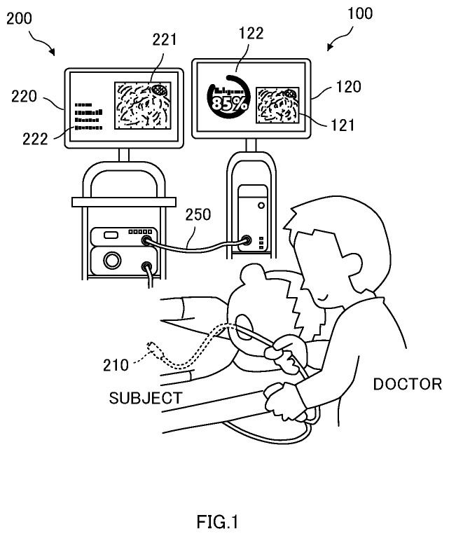

FIG. 1 is a diagram showing a manner of an endoscopic examination performed using an endoscope system 200 and an examination support device 100 according to a present embodiment. The endoscope system 200 and the examination support device 100 are both installed in a consultation space. The present embodiment assumes a case where, of inside of a body of a subject, a stomach is examined. The endoscope system 200 includes a camera unit 210, and as shown in the drawing, the camera unit 210 is inserted into the stomach of a subject who is lying down, through a mouth, and transmits, to a system main body, an image signal of an image obtained by capturing an inside of the stomach. Insertion of the camera unit 210 into the stomach and an image capturing operation are performed by a doctor.

The endoscope system 200 includes a system monitor 220 configured by a liquid crystal panel, for example, and processes the image signal transmitted from the camera unit 210 and displays the same as a captured image 221 that can be viewed, on the system monitor 220. Furthermore, the endoscope system 200 displays examination information 222 including subject information, camera information of the camera unit 210, and the like on the system monitor 220.

The examination support device 100 is connected to the endoscope system 200 by a connection cable 250. The endoscope system 200 transmits a display signal that is transmitted to the system monitor 220, also to the examination support device 100 via the connection cable 250. That is, the display signal in the present embodiment is an example of an output signal that is output by the endoscope system 200 to an external device. The examination support device 100 includes a display monitor 120 configured by a liquid crystal panel, for example, and extracts an image signal corresponding to the captured image 221 from the display signal that is transmitted from the endoscope system 200, and displays the same as a captured image 121 that can be viewed, on the display monitor 120. Furthermore, the examination support device 100 generates and analyzes image data of the captured image 121, outputs diagnosis support information 122, and displays the same on the display monitor 120. That is, the doctor is able to sequentially check the captured image 121 and the diagnosis support information 122 synchronously with progress of examination.

Moreover, the examination support device 100 supports a screening examination that is performed using the endoscope system 200. More specifically, whether an image that should be acquired that is determined in relation to the screening examination is acquired or not is determined, and a warning is issued in a case where there is still an image that is yet to be acquired, at a timing of removal of the camera unit 210 from an examination part (in the present embodiment, the stomach). Specific processes will be described later in detail.

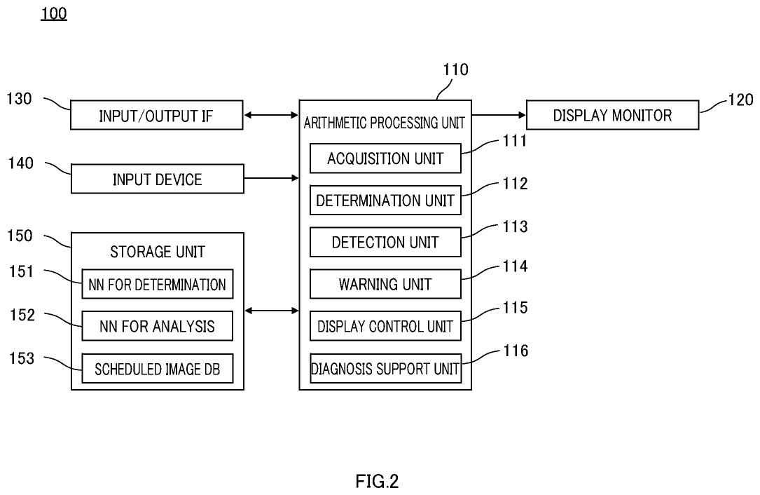

FIG. 2 is a hardware configuration diagram of the examination support device 100. The examination support device 100 mainly includes an arithmetic processing unit 110, the display monitor 120, an input/output interface 130, an input device 140, and a storage unit 150. The arithmetic processing unit 110 is a processor (CPU: Central Processing Unit) that performs processes of controlling the examination support device 100 and executing programs. The processor may operate in conjunction with an arithmetic processing chip such as an application specific integrated circuit (ASIC) or a graphics processing unit (GPU). The arithmetic processing unit 110 performs various processes related to supporting of examination by reading an examination support program that is stored in the storage unit 150.

As described above, the display monitor 120 is a monitor including a liquid crystal panel, for example, and the display monitor 120 displays the captured image 121, the diagnosis support information 122 and the like in a viewable manner. The input/output interface 130 is a connection interface that includes a connector for connecting the connection cable 250, and that is for exchanging information with an external appliance. The input/output interface 130 includes a LAN unit, for example, and takes in the examination support program and update data for a neural network 152 for analysis described later from an external appliance and transfers the same to the arithmetic processing unit 110.

The input device 140 is a keyboard, a mouse, or a touch panel superimposed on the display monitor 120, for example, and a doctor or an assistant operates the same to change settings of the examination support device 100 and to input information necessary for examination.

The storage unit 150 is a non-volatile storage medium, and is a hard disk drive (HDD), for example. The storage unit 150 is capable of storing, in addition to programs for controlling the examination support device 100 and for executing processes, various parameter values to be used for control and calculation, functions, display element data, look-up tables, and the like. In particular, the storage unit 150 stores a neural network 151 for determination, the neural network 152 for analysis, and a scheduled image database 153.

The neural network 151 for determination is a trained model for determining, when image data captured by the camera unit 210 is input, whether a corresponding image matches any of scheduled images that should be acquired. The neural network 152 for analysis is a trained model for calculating, when image data captured by the camera unit 210 is input, a probability of existence of a lesion in a corresponding image. The scheduled image database 153 is a database collecting samples of images that should be acquired that are determined in relation to a screening examination, and guidance information for capturing the images. Additionally, the storage unit 150 may be configured from a plurality of pieces of hardware, and for example, a storage medium for storing programs, and a storage medium for storing the neural network 151 for determination and the like may be configured by separate pieces of hardware.

The arithmetic processing unit 110 also serves a role of an arithmetic functional unit that performs various calculations according to processes instructed by the examination support program. The arithmetic processing unit 110 may function as an acquisition unit 111, a determination unit 112, a detection unit 113, a warning unit 114, a display control unit 115, and a diagnosis support unit 116. The acquisition unit 111 processes a display signal that is sequentially transmitted from the endoscope system 200, and thus reproduces and acquires the captured image captured by the camera unit 210. The determination unit 112 determines whether the captured image that is acquired by the acquisition unit 111 matches any of scheduled images that should be acquired that are set in advance. The detection unit 113 detects a timing of removal of the camera unit 210 from the examination part, based on the captured image that is acquired by the acquisition unit 111. The warning unit 114 checks whether there remains a scheduled image for which match is not determined by the determination unit 112, at the timing detected by the detection unit 113, and in the case where there remains such a scheduled image, the warning unit 114 issues a warning to the effect. The display control unit 115 controls display on the display monitor 120 by generating a display signal of a display screen to be displayed on the display monitor 120 and transmitting the display signal to the display monitor 120. The diagnosis support unit 116 inputs the captured image to the neural network 152 for analysis read from the storage unit 150 and causes the probability of existence of a lesion to be calculated, and generates diagnosis support information.

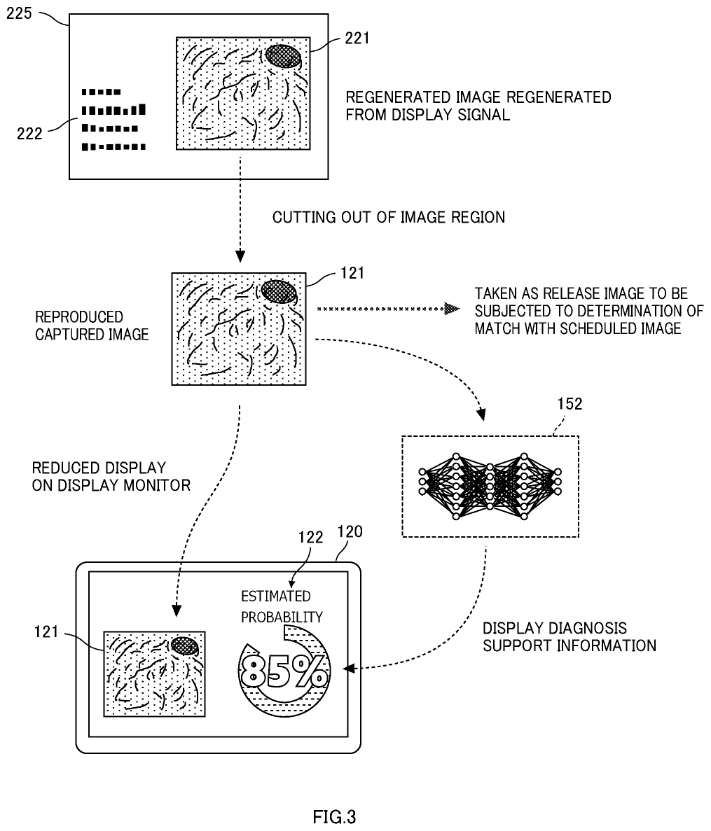

FIG. 3 is a diagram describing a process up to generation of a display screen on the display monitor 120 from a display signal that is received at the time of examination. A regenerated signal image 225 that is obtained by the acquisition unit 111 acquiring and developing a display signal transmitted from the endoscope system 200 is the same as a display image that is displayed on the system monitor 220 of the endoscope system 200. As described above, the regenerated signal image 225 includes the captured image 221 and the examination information 222. The examination information 222 here is assumed to be text information, but may also include information other than the text information, such as computer graphics.

In the case where the examination support device 100 is used by being connected to an existing endoscope system 200, a dedicated signal convenient for the examination support device 100 is not received, but a general-purpose image signal that the endoscope system 200 provides to an external device, such as the display signal, is used. The general-purpose image signal includes an image signal corresponding to the captured image captured by the camera unit 210, but also includes image signals corresponding to information associated with examination and a GUI. To enable the examination support device 100 to analyze the captured image captured by the camera unit 210 and to perform determination of match/non-match with a scheduled image that should be acquired, an image region in the captured image has to be appropriately cut out from an image region in the image signal received from the endoscope system 200 and image data of the captured image has to be regenerated. In the present embodiment, the doctor or an assistant specifies the image region in the captured image using the input device 140, at the time of start of use of the examination support device 100.

The acquisition unit 111 cuts out, as the captured image 121, the image region that is specified, from the regenerated signal image 225. The captured image 121 can basically be said to be an image that is obtained by reproducing, by the examination support device 100, the captured image captured by the camera unit 210. In the case where captured image data of the captured image 121 that is reproduced in the above manner is determined to be a release image, the acquisition unit 111 transfers the captured image 121 to the determination unit 112, the diagnosis support unit 116, and the display control unit 115. Determination of whether the captured image data is a release image or not, and determination of match/non-match by the determination unit 112 will be described later.

The diagnosis support unit 116 inputs to the neural network 152 for analysis, the captured image data that is received, causes an estimated probability of existence of a lesion in the image to be calculated, and transfers a result to the display control unit 115. The display control unit 115 displays the captured image data received from the acquisition unit 111 and diagnosis support information including the estimated probability received from the diagnosis support unit 116 on the display monitor 120 by developing and arranging the same according to a display mode set in advance. More specifically, as shown in a bottom section in FIG. 3, for example, the captured image 121 is reduced and arranged on a left side, and the diagnosis support information 122 is arranged on a right side while being segmented into elements including numerical value information indicating the estimated probability, title text, and circle graph graphics. Additionally, such a display mode is merely an example, and each display element is not necessarily displayed during examination.

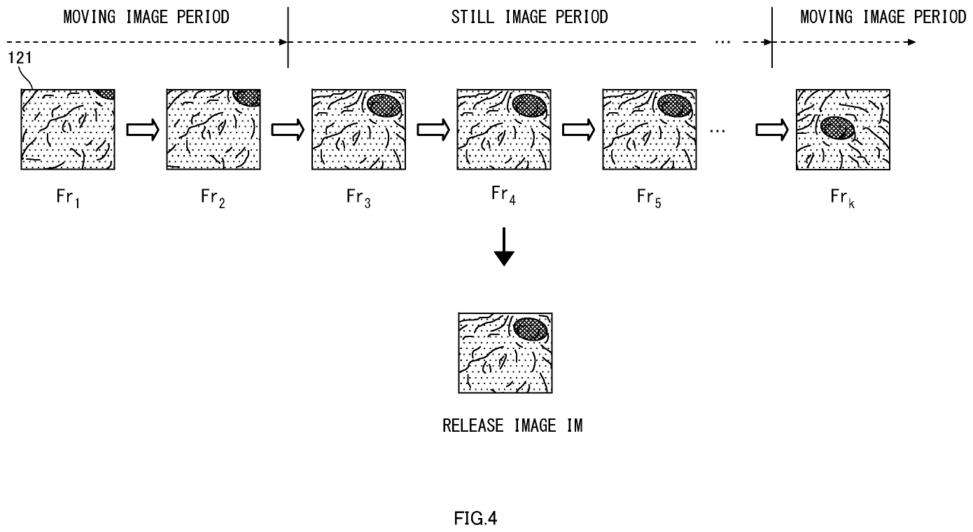

FIG. 4 is a diagram describing a change in the captured image 121 that is reproduced. The examination support device 100 receives the display signal at a cycle of 60 fps, for example, and each time, the acquisition unit 111 cuts out the image region from the regenerated signal image 225 and generates the captured image 121 in the manner described above. The captured image 121 that is sequentially generated may be treated as a frame image Fr that may change over time. FIG. 4 shows frame images Fr₁, Fr₂, Fr₃, Fr₄, Fr₅, . . . , Frₖ that are sequentially generated.

The camera unit 210 successively performs capturing, and sequentially transmits an image signal as the frame image to the endoscope system 200. While display signals including such image signals are being received from the endoscope system 200, the acquisition unit 111 sequentially generates the frame images Fr that correspond to the captured images of the camera unit 210 and that change over time. This period is taken as a moving image period.

The doctor views the captured image 221 that is displayed on the system monitor 220 or the captured image 121 that is displayed on the display monitor 120, and presses a release button provided on an operation portion of the camera unit 210 at a timing when the image data is desired to be saved. The endoscope system 200 converts the image signal captured by the camera unit 210 into image data of a still image and performs a recording process at a timing when the release button is pressed. Then, the image signal in the display signal to be output to the examination support device 100 is fixed to an image signal corresponding to the still image for a certain period of time (such as 3 seconds).

While the display signal including such an image signal is being received from the endoscope system 200, the acquisition unit 111 sequentially generates the frame images Fr corresponding to the still image generated by pressing of release. This period is taken as a still image period. The still image period continues until the certain period of time when the image signal is fixed by the endoscope system 200 is elapsed, and then, the moving image period is started again. In the example in FIG. 4, up to the frame image Fr₂ is the moving image period, and the still image period begins from the frame image Fr₃, and then, the moving image period is started again from the frame image Frₖ.

In the case where a release signal is included in the display signal received from the endoscope system 200, the acquisition unit 111 may refer to the release signal, and may perceive that the still image period starts from the frame image Fr₃. In the case where the release signal is not included in the display signal received from the endoscope system 200, a difference between adjacent frame images may be detected, and the still image period may be perceived in the case where an integrated amount falls below a threshold, for example.

In the case where the generated frame images Fr are determined based on the perception described above to be the frame images in the still image period, the acquisition unit 111 takes one of the frame images to be a release image IM and transfers the same to the determination unit 112 and the diagnosis support unit 116 in the manner described with reference to FIG. 3. As described above, the diagnosis support unit 116 performs, in relation to the release image IM, calculation for diagnosis support. The determination unit 112 determines whether the release image IM matches any of the scheduled images that should be acquired that are set in advance.

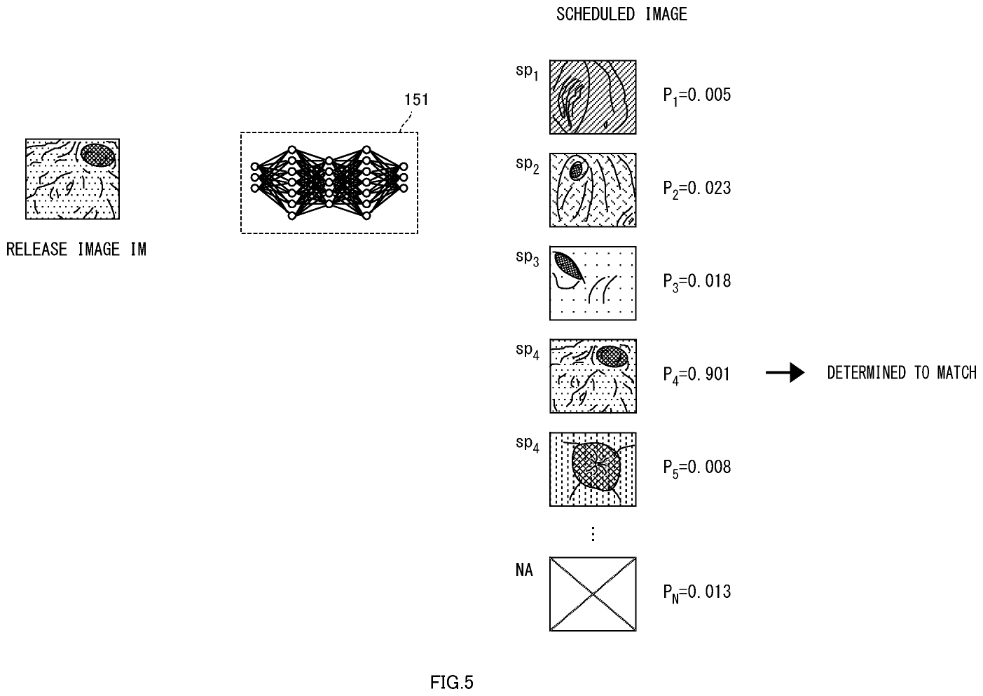

FIG. 5 is a diagram describing a process of match determination by the determination unit 112. The determination unit 112 inputs the release image IM received from the acquisition unit 111 to the neural network 151 for determination read out from the storage unit 150, and causes a probability of match with each of the scheduled images that should be acquired to be calculated.

The neural network 151 for determination is created in advance by supervised learning using a substantial amount of image data where a ground truth indicating a scheduled image is associated with each of the scheduled images that should be acquired. More specifically, “1” (=ground truth) is given to a training image corresponding to a k-th scheduled image spₖ, as a probability Pₖ of being the scheduled image spₖ. A substantial number of such training images is prepared for the k-th scheduled image spₖ. In the case where the number of schedules images is n, the neural network 151 for determination is created by learning the training images that are prepared for each k, k being one of 1 to n. The neural network 151 for determination created in this manner is stored in the storage unit 150, and is read out by the determination unit 112 as appropriate to be used.

As shown in FIG. 5, when the release image IM is input, the neural network 151 for determination calculates and outputs each probability Pₖ of being the scheduled image spₖ. More specifically, output is performed in such a way that the probability of being a first scheduled image sp₁ is P₁=0.005, the probability of being a second scheduled image sp₂ is P₂=0.023, the probability of being a third scheduled image sp₃ is P₃=0.018, the probability of being a fourth scheduled image sp₄ is P₄=0.901, and the probability of not belonging to any scheduled image is P_{N}=0.013. The determination unit 112 selects, from the probabilities that are output, one that is equal to or greater than a threshold and that indicates a maximum value, and determines the scheduled image corresponding to the probability to be the image that matches the release image IM. The threshold is set in advance to a value (for example, 0.8) by which there is assumed to be no error in the determination. In the example in FIG. 5, the release image IM is determined to match the scheduled image sp₄.

In the case where the release image IM is determined to match any of the scheduled images, the determination unit 112 associates determination information about the scheduled image that is determined to match, with the release image IM. More specifically, information indicating a position in an order of the scheduled images is written in header information of the image data of the release image IM. After performing writing in the header information, the determination unit 112 stores the image data in the storage unit 150. Additionally, association of the determination information is not limited to writing in the header information of the image data, and may alternatively be managed by creating a management list or the like separately from the image data. Furthermore, association is not limited to be performed with the image data of the release image IM generated by the acquisition unit 111, and may alternatively be performed with the image data of the original captured image recorded by the endoscope system 200. Either way, it suffices if the result of match determination is associated with the captured image that is generated according to pressing of release.

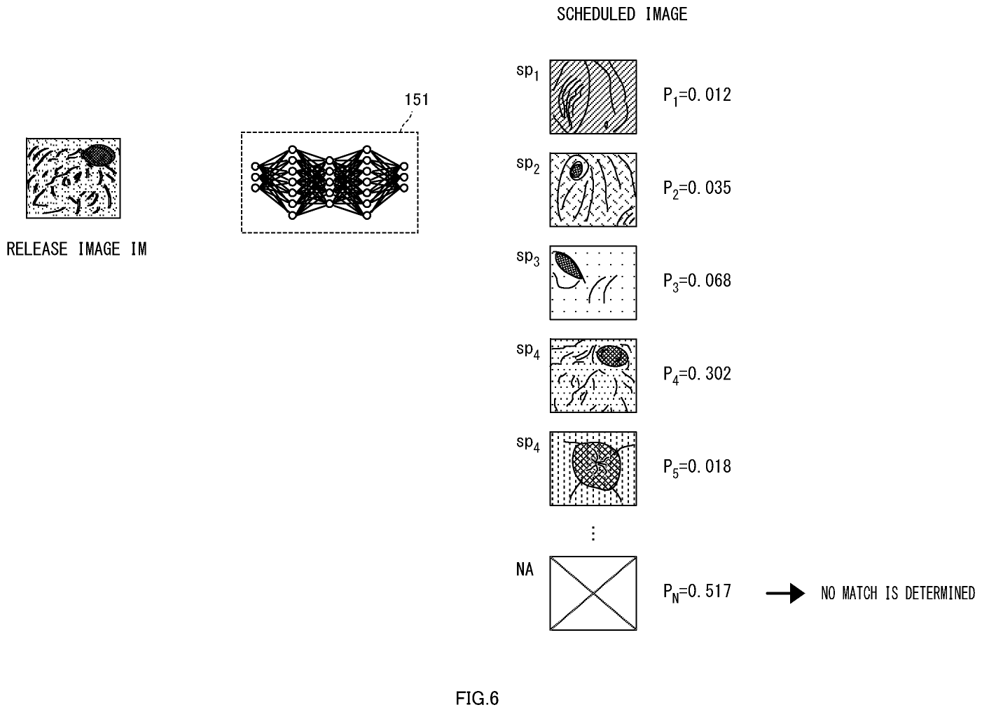

FIG. 6 is a diagram further describing the process of match determination by the determination unit 112. As shown in the drawing, in the case where the probability P_{N} takes the maximum value, the determination unit 112 determines that the release image IM does not match any of the scheduled images. To “not match any of the scheduled images” includes, in addition to a case where the release image IM is not obtained by capturing one of a plurality of parts inside a stomach that are set in advance, a case where one of the plurality of parts that are set in advance is captured but the image is greatly blurred or is out of focus. That is, a low-quality release image IM is determined to not match any of the scheduled images.

Here, in the case where the release image IM is determined to match any of the scheduled images, the determination unit 112 may also determine whether the image satisfies a quality standard set in advance. In this case, the determination unit 112 may analyze frequency components of the release image IM as a target image and may determine that the release image IM is not clear in a case where a high-frequency component at or higher than a reference value is not included, or may determine inappropriate exposure in a case where a luminance distribution is biased to either a bright side or a dark side by a reference proportion or more.

In the case where the release image IM is determined by the determination unit 112 not to satisfy the quality standard, the determination result is transferred to the warning unit 114, and the warning unit 114 displays, on the display monitor 120 via the display control unit 115, a warning indicating that the image does not satisfy the quality standard. Additionally, the warning that is issued by the warning unit 114 here is a warning of a mode different from a warning, described later, indicating that there remains an image that is not yet acquired.

Criteria are set for screening examinations that use an endoscope system such that various sections of an examination part (in the present embodiment, a stomach) are captured from various angles and at various angles of view to be recorded as still images for subsequent detailed imaging study. The scheduled image of the present embodiment is assumed to be a still image that is set in the criteria. However, due to lack of experience or an error of a doctor manipulating an endoscope, a still image that should be recorded may possibly be forgotten to be taken or may be unclear. Insertion/removal of an endoscope from inside a body can be somewhat uncomfortable to a subject, and redoing of examination should be avoided as much as possible. Accordingly, the examination support device 100 according to the present embodiment detects a timing of removal of the camera unit 210 from the stomach, checks whether there is still an image to be acquired at the timing, and issues a warning to the doctor in the case where there remains such an image. First, a process of detecting the timing of removal of the camera unit 210 from the stomach will be described.

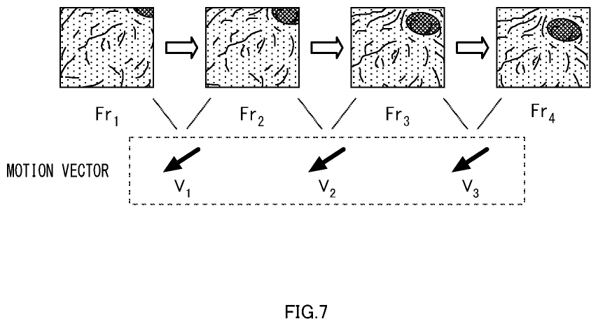

FIG. 7 is a diagram describing a process of removal detection by the detection unit 113. A timing when the doctor pulls out the camera unit 210 from the examination part is assumed to be in the moving image period described with reference to FIG. 4. In the case where the frame images Fr that are generated are determined to be the frame images in the moving image period, the acquisition unit 111 sequentially transfers the frame images Fr to the detection unit 113.

When successive frame images Fr are received, the detection unit 113 compares adjacent frame images and successively calculates a motion vector indicating a change in a figure. In the example in FIG. 7, in the case where frame images Fr₁, Fr₂, Fr₃, Fr₄ are sequentially received, a motion vector V₁ is calculated by comparing the frame images Fr₁ and Fr₂, a motion vector V₂ is calculated by comparing the frame images Fr₂ and Fr₃, and a motion vector V₃ is calculated by comparing the frame images Fr₃ and Fr₄.

When the doctor starts to remove the camera unit 210 from the examination part, a figure in the frame images Fr continuously flows in one direction. Accordingly, the detection unit 113 determines a time when the calculated motion vectors V indicate a same direction over a specific period of time and all have a size that is equal to or greater than a threshold to be a timing of start of removal of the camera unit 210 from the examination part. Here, the specific period of time, an allowable deviation range regarding the same direction, and the threshold regarding the size of the vector that are set are determined through trial and error by taking into account a frame rate of the frame image Fr, the number of pixels, a removal speed that is assumed, and the like. Additionally, calculation of the motion vector V may be performed with respect not only to continuous frame images but also to frame images that are next to each other with several frames in-between.

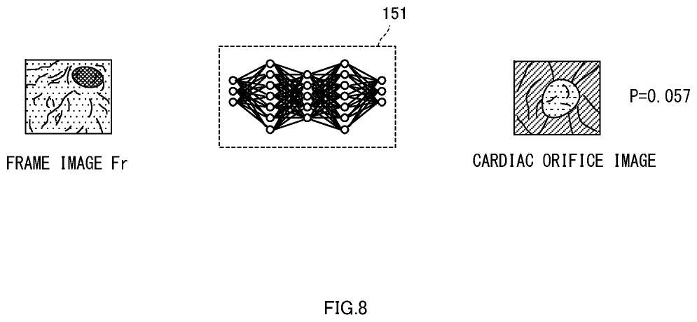

FIG. 8 is a diagram describing another process of removal detection by the detection unit 113. When the doctor pulls out the camera unit 210 from the stomach that is the examination part, the camera unit 210 passes through a cardiac orifice, which is a connection part between an esophagus and the stomach. Accordingly, the detection unit 113 monitors the frame images Fr received from the acquisition unit 111, and determines a time when a shape of the cardiac orifice is detected in the image as the timing of removal of the camera unit 210 from the stomach.

More specifically, the detection unit 113 reads out the neural network 151 for determination from the storage unit 150, and inputs the frame image Fr received from the acquisition unit 111. The neural network 151 for determination that is used here is trained using, in addition to the scheduled image described above, a cardiac orifice image that is captured when the camera unit 210 passes through the cardiac orifice, and a probability of match between the frame image Fr that is input and the cardiac orifice image is also calculated. When a probability P regarding the frame image Fr that is input is calculated by the neural network 151 for determination, the detection unit 113 checks whether the probability P exceeds a threshold (for example, 0.8) or not. In the case where the threshold is exceeded, the frame image Fr is determined to be the cardiac orifice image, and the time is determined to be the timing of removal of the camera unit 210 from the stomach. Additionally, the probability that is calculated for the frame image Fr in the drawing is P=0.057, and the frame image Fr is not determined to be the cardiac orifice image. Additionally, a part different from the cardiac orifice may also be selected as long as the part is a part that the camera unit 210 inevitably passes through at the time of being removed. For example, the esophagus or a mouth may be selected instead of the cardiac orifice. Furthermore, pulling out of the camera unit 210 may be determined in the case where a shape of the esophagus is detected after the shape of the cardiac orifice is detected.

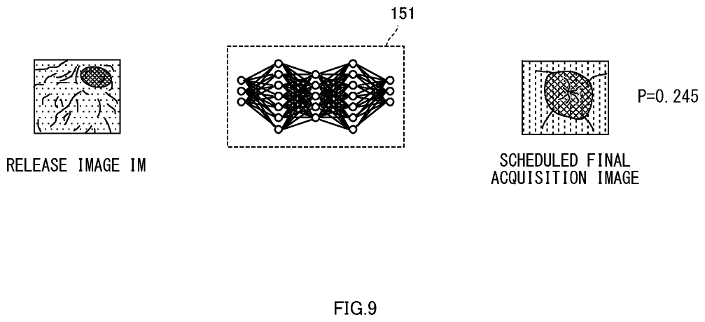

FIG. 9 is a diagram describing further another process of removal detection by the detection unit 113. As described above, in the screening examinations that use an endoscope system, scheduled images that should be acquired are determined in advance, and an order of acquisition may be sometimes also determined. Accordingly, the detection unit 113 monitors whether the release image IM for which match is determined by the determination unit 112 matches a final acquisition image that is the scheduled image that is to be acquired last.

More specifically, the detection unit 113 receives the determination result from the determination unit 112 as appropriate, and determines a time when it is checked that the determination result indicates match with the final acquisition image to be the timing when the camera unit 210 is removed from the examination part (in the present embodiment, the stomach). Additionally, the probability that is calculated for the release image IM shown in the drawing is P=0.245, and the release image IM is not determined to be a scheduled final acquisition image. In the case where the examination part is the stomach, an image of a pylorus, which is a connection part to a duodenum and which is located at a deepest part of the stomach, is set as the scheduled final acquisition image, for example.

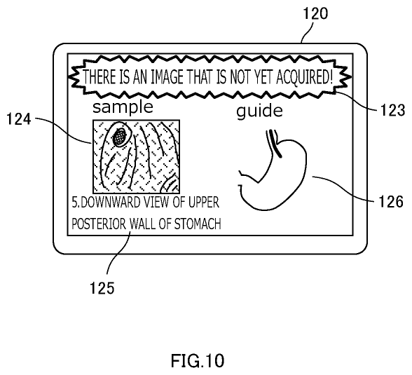

When removal of the camera unit 210 is detected, the detection unit 113 immediately notifies the warning unit 114 of the same. A notification of the timing of removal of the camera unit 210 from the examination part is thus actually issued. When the notification of removal detection is received from the detection unit 113, the warning unit 114 checks whether there remains a scheduled image for which match is not yet determined by the determination unit 112, among the scheduled images that should be acquired. More specifically, check is performed by referring to pieces of image data that are stored in the storage unit 150 up to then and the determination information that is associated with each piece of the image data. Then, in the case where it is checked that there remains such a scheduled image, a warning is immediately issued. FIG. 10 is an example of a warning screen that is displayed on the display monitor 120 in a case where there remains such a scheduled image.

The warning screen includes warning text 123, a sample image 124, text information 125, and a guidance 126. The warning text 123 is “there is an image that is not yet acquired!”, for example, and is a warning sentence indicating that an image that should be acquired is not acquired. The warning text 123 may be accompanied by flashing display for emphasis or graphics. The sample image 124 is an example of an image that should be acquired but that is forgotten by the doctor, and is selectively read out from the scheduled image database 153 stored in the storage unit 150 to be displayed. The text information 125 includes a name of a specific section of the examination part corresponding to the scheduled image that should be acquired, and information about an observation direction.

The guidance 126 is support information about how a scheduled image that is not yet acquired can be captured and acquired, and the guidance 126 describes, using graphics, a position, in the stomach that is the examination part, to which a distal end portion of the camera unit 210 is to be guided. Guidance information for generating the guidance 126 is stored in the scheduled image database 153 in association with the sample image 124. Additionally, in the case where there remains a plurality of scheduled images that should be acquired, display of the sample image 124, the text information 125, and the guidance 126 corresponding to respective scheduled images may be performed while sequentially switching the display, or display may be performed in the form of a list, next to each other. Additionally, the warning unit 114 may perform a process of issuing a warning sound via a speaker, for example, in addition to displaying a warning on the display monitor 120.

As described above, when the warning unit 114 immediately issues a warning in the case where there remains a scheduled image that should be acquired, the doctor may more likely become aware of the fact before pulling out the camera unit 210 from the mouth of the subject, and may continue on with capturing that is insufficient. When the doctor continues on with capturing in this manner, discomfort of the subject can be reduced than in a case where examination is performed again after the camera unit 210 is pulled out from the mouth.

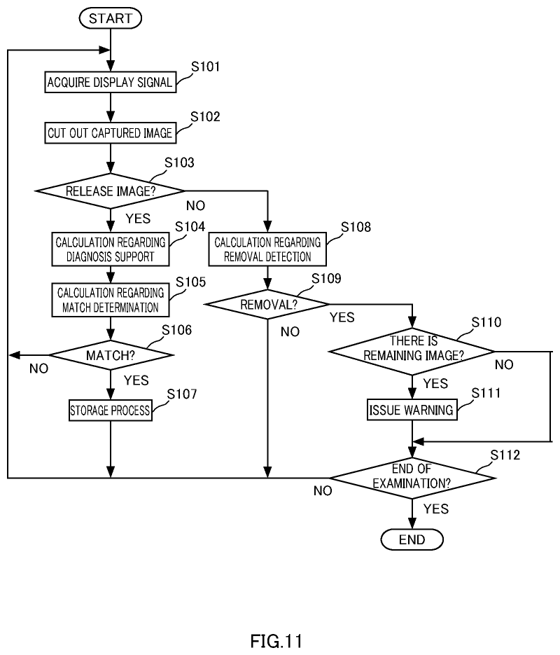

Next, a procedure of an examination support method that uses the examination support device 100 will be described. FIG. 11 is a flowchart describing a procedure performed by the arithmetic processing unit 110. A case of performing removal detection will be described here with reference to FIG. 7. The flow is started when the camera unit 210 is inserted into the body of the subject from the mouth, for example.

The acquisition unit 111 acquires the display signal from the endoscope system 200 in step S101, and cuts out the captured image from the display image obtained by developing the display signal and generates a current frame image in step S102. Then, in step S103, an earlier frame image and the current frame image are compared and whether the current frame image is a release image in the still image period or not is determined in the manner described with reference to FIG. 4. In the case where the current frame image is determined to be the release image, the acquisition unit 111 transfers the current frame image to the determination unit 112 and the diagnosis support unit 116, and proceeds to step S104. In the case where the current frame image is not determined to be the release image, the current frame image is transferred to the detection unit 113, and step S108 is performed next.

In step S104 following step S103, the diagnosis support unit 116 calculates the estimated probability in the manner described above for the release image received from the acquisition unit 111, and transfers the result to the display control unit 115. The display control unit 115 displays the examination information on the display monitor 120 according to a display mode as shown in the bottom section in FIG. 3. As described with reference to FIGS. 5 and 6, in step S105, the determination unit 112 inputs the release image received from the acquisition unit 111 to the neural network 151 for determination, and causes the probability that the release image matches a scheduled image that should be acquired to be calculated. Then, in step S106, whether the release image matches any of the scheduled images that should be acquired is determined based on the probabilities that are calculated. In the case where match is determined, step S107 is performed, and the determination information about the scheduled image with respect to which match is determined is stored in the storage unit 150, in association with the release image. When a storage process is completed, the process returns to step S101. In the case where match is not determined in step S106 with respect to any of the scheduled images that should be acquired, step S107 is skipped, and the process returns to step S101. Additionally, the process in step S104 related to diagnosis support, and the processes from step S105 to step S107 related to match determination may be reversed in order, or may be performed in parallel.

In step S108 following step S103, the detection unit 113 compares the current frame image received from the acquisition unit 111 and a frame image received in the past and calculates the motion vector, as described with reference to FIG. 7. Then, a change in the motion vector is checked by referring also to the motion vectors accumulated up to then. The detection unit 113 proceeds to step S109, checks a change in the motion vector, and then if a shape in the frame images is detected to continuously flow in a certain direction, the detection unit 113 determines the timing of start of removal of the camera unit 210 from the examination part, issues a notification to the effect to the warning unit 114, and proceeds to step S110. The process returns to step S101 when such a state is not detected.

In step S110, the warning unit 114 checks whether there still remains a scheduled image with respect to which match is not determined by the determination unit 112, among the scheduled images that should be acquired. In the case where it is determined that there remains such an image, step S111 is performed, and the warning described with reference to FIG. 10 is issued, and step S112 is then performed. In the case where it is determined that there remains no such image, step S111 is skipped and step S112 is performed. In step S112, the arithmetic processing unit 110 checks whether an instruction to end examination is received or not, and step S101 is performed again when the instruction to end is not received, and the series of processes is ended when the instruction to end is received.

In the present embodiment described above, a case is assumed where the endoscope system 200 and the examination support device 100 are connected by the connection cable 250, but wireless connection may also be adopted instead of wired connection. Furthermore, an embodiment is described where the endoscope system 200 outputs a display signal to outside, and where the examination support device 100 uses the display signal, but in the case where an image signal that is provided to an external device by the endoscope system 200 includes the image signal of a captured image that is captured by the camera unit 210, the format of an output signal is irrelevant. Furthermore, in the case where the endoscope system 200 continuously provides to outside only the captured signals, the process of cutting out on the side of the examination support device 100 and the like are not necessary.

Moreover, the examination support device 100 may be embedded as a part of the endoscope system 200. Also in this case, the captured signal transmitted from the camera unit 210 may be directly processed, and the process of cutting out and the like are not necessary.

Furthermore, in the present embodiment described above, an example where the examination part is the stomach is described, but the endoscope system 200 does not have to be specialized for stomach examination, and may also be used for examination of other parts. For example, the examination support device 100 that is connected to the endoscope system 200 for examining a duodenum supports the screening examination for the duodenum such that scheduled images that should be acquired are all acquired. Moreover, in the present embodiment described above, a description is given assuming that the camera unit 210 of the endoscope system 200 is a flexible endoscope, but the configuration and processes of the examination support device 100 are no different even when the camera unit 210 is a rigid endoscope.

What is claimed is:

1. An examination support device comprising:
an acquisition unit that sequentially acquires a captured image captured by a camera unit inserted in a body of a subject;
a determination unit that determines whether the captured image acquired by the acquisition unit matches any of scheduled images that should be acquired;
a detection unit that detects a timing of removal of the camera unit from an examination part, based on the captured image acquired by the acquisition unit; and
a warning unit that issues a warning in a case where there remains, at the timing detected by the detection unit, the scheduled images for which match is not determined by the determination unit.

2. The examination support device according to claim 1, wherein
the determination unit determines whether any of the scheduled images is matched, in a case where the captured image is detected to be a still image, and
the detection unit detects the timing based on a frame image that is the captured image that changes sequentially.

3. The examination support device according to claim 2, wherein the detection unit takes, as the timing, a time when a figure of a cardiac orifice of a stomach as the examination part is detected in the frame image.

4. The examination support device according to claim 2, wherein the detection unit takes, as the timing, a time when continuous flow of the frame image in a certain direction is detected.

5. The examination support device according to claim 1, wherein the detection unit takes, as the timing, a time when the captured image is determined by the determination unit to match a final image that is set in advance among the scheduled images.

6. The examination support device according to claim 1, wherein the warning unit also outputs information about a remaining image that is yet to be acquired among the scheduled images.

7. The examination support device according to claim 6, wherein the warning unit also outputs a guidance for acquiring the image that is yet to be acquired.

8. The examination support device according to claim 1, wherein, in a case where the captured image is determined to match any of the scheduled images, the determination unit associates information about the scheduled image for which match is determined with the captured image.

9. The examination support device according to claim 1, wherein
in a case where the captured image is determined to match any of the scheduled images, the determination unit further determines whether the captured image satisfies a quality standard that is set in advance, and
in a case where the captured image is determined by the determination unit not to satisfy the quality standard, the warning unit issues a warning different from the warning that is issued in a case where there remains the scheduled images.

10. The examination support device according to claim 1, wherein the acquisition unit acquires the captured image by receiving and processing an output signal output from an endoscope system.

11. An examination support method comprising:
an acquisition step of sequentially acquiring a captured image captured by a camera unit inserted in a body of a subject;
a determination step of determining whether the captured image acquired in the acquisition step matches any of scheduled images that should be acquired;
a detection step of detecting a timing of removal of the camera unit from an examination part based on the captured image acquired in the acquisition step; and
a warning step of issuing a warning in a case where there remains, at the timing detected in the detection step, the scheduled images for which match is not determined in the determination step.

12. A storage medium storing an examination support program for causing a computer to perform:
an acquisition step of sequentially acquiring a captured image captured by a camera unit inserted in a body of a subject;
a determination step of determining whether the captured image acquired in the acquisition step matches any of scheduled images that should be acquired;
a detection step of detecting a timing of removal of the camera unit from an examination part based on the captured image acquired in the acquisition step; and
a warning step of issuing a warning in a case where there remains, at the timing detected in the detection step, the scheduled images for which match is not determined in the determination step.
